(19) United States
(12) Patent Application Publication
(10) Pub. No.: US 2012/0064522 A1
(43) Pub. Date: Mar. 15, 2012

(54) METHODS FOR DETERMINING GENE-ALPHA TOCOPHEROL INTERACTIONS

(76) Inventors: Rosalynn D. Gill, Boulder, CO (US); Robert F. Grimble, Southampton (GB)

(21) Appl. No.: 13/201,907

(22) PCT Filed: Feb. 18, 2010
(86) PCT No.: PCT/US10/24611

§ 371 Date: Oct. 24, 2011

Related U.S. Application Data
(60) Provisional application No. 61/153,606, filed on Feb. 18, 2009.

(51) Int. Cl. C12Q 1/68

(52) U.S. Cl. 435/6.11

(57) ABSTRACT

Method for predicting the change in the level of at least one pro-inflammatory cytokine in an individual due to alpha tocopherol supplementation are provided, as well as methods for predicting the response of an individual to alpha tocopherol supplementation, and methods of nutrigenetic screening of an individual.

### FIELD OF THE INVENTION

The present invention relates to methods for predicting metabolic responses to dietary factors and to providing dietary and lifestyle advice based on gene-nutrient interactions, based on genetic polymorphisms correlated with a change in serum pro-inflammatory cytokine levels upon supplementation with Vitamin E.

### BACKGROUND OF THE INVENTION

Vitamin E, especially alpha tocopherol (αT), exhibits antioxidant as well as anti-inflammatory activity and inhibits several biological events involved in atherogenesis (1). Such events include lower levels of c-reactive proteins and urinary isoprostanes, decreased levels of prostaglandin synthesis and of platelet aggregation (see (2) for a review).

In vitro studies have demonstrated the superior antioxidant properties of αT in the prevention of LDL lipid peroxidation due to its lipid solubility and preferential incorporation into lipoproteins (3). Overall, several lines of evidence support a relationship between low αT levels and the development of atherosclerosis (for a review, see (4)). The anti-inflammatory significance of αT has also been highlighted in animal models by showing increased basal oxidative stress and inflammatory status among knock-out mice who lack the liver protein responsible for incorporating αT into very-low-density lipoprotein i.e., the α-tocopherol transfer protein (5).

Although the studies carried out with cell culture and animal models suggest that αT has promising antiatherosclerotic effects, the results of its supplementation in humans are controversial. In 1993 a report from the Nurses' Health Study, a prospective cohort of more than 80,000 U.S. nurses (6) showed a 30% reduction of major coronary heart disease in subjects who took vitamin E supplements after adjustment for age, smoking status and intake of carotene and vitamin C. Subsequently however a cohort study of 5133 Finnish residents followed for 14 years, found no risk reduction due to vitamin E supplementation (7). The Physicians' Health Study conducted among more than 80,000 US healthy male physician also showed no association of vitamin E supplement use and reduction of coronary disease (8).

Several interventional trials have also investigated the role of α-tocopherol supplementation on cardiovascular and inflammatory end-points and yielded mixed results (9). Either vitamin E did not appear beneficial or was only beneficial for certain outcomes (such non fatal myocardial infarction) and not others (fatal coronary heart disease), or was found to be beneficial only for certain types of patients (e.g. those with compromised antioxidant activity). (10).

Oxidative stress, induced under a variety of conditions, is known to lead to the enhanced liberation of proinflammatory chemokines and cytokines (14). A decrease in oxidative stress, therefore should be conducive to a decreased release of pro-inflammatory cytokines. The results from large clinical trials with αT on the risk of cardiovascular disease—which is strongly affected by inflammation, have been mostly negative (15-17). Other studies in subjects with enhanced oxidative stress associated with end-stage renal disease (18), in subjects with elevated cholesterol (19), and, in combination with vitamin C, in subjects undergoing transplant (20) were more promising. A recent study among subjects with type 2 diabetes (21) found that markers of oxidative stress decreased as a result of α T and gamma-tocopherol supplementation but markers of inflammation such as c-reactive protein, monocyte chemoattractant protein-1, TNF-α and IL-6 showed no improvement.

Inconsistency among these studies that were all well designed and carried out in large study samples cannot be easily explained. Some of the reasons suggested by authors to explain such data include inadequate selection of subjects (by gender, vitamin E status, etc.) or of the dose, timing of intake, and chemical form of tocopherol. One aspect which has not been addressed is the influence that an individual's genetic background may have on the response to αT supplementation.

### BRIEF DESCRIPTION OF THE FIGURES

FIG. 1A-1D shows changes in cytokine serum levels after 6 weeks of αT supplementation in healthy males.

FIG. 2A shows the change in TNFα serum levels after 6 weeks of αT supplementation for genotypes for the IL10-1082 polymorphism.

FIG. 2B shows the change in TNFα serum levels after 6 weeks of αT supplementation for genotypes for the TNF-238 polymorphism.

FIG. 3A shows the change in IL-6 serum levels after 6 weeks of αT supplementation for genotypes for the GSTP1 313 polymorphism.

FIG. 3B shows the change in IL-6 serum levels after 6 weeks of αT supplementation for genotypes for the SOD2-28 polymorphism.

FIG. 4A shows the change in IL-1 serum levels after 6 weeks of αT supplementation for genotypes for the GSTP1 313 polymorphism.

FIG. 4B shows the change in IL-1 serum levels after 6 weeks of αT supplementation for genotypes for the IL10-592 polymorphism.

FIG. 4C shows the change in IL-1 serum levels after 6 weeks of αT supplementation for genotypes for the IL10-819 polymorphism.

FIG. 4D shows the change in IL-1 serum levels after 6 weeks of αT supplementation for genotypes for the IL10-1082 polymorphism.

### SUMMARY OF THE INVENTION

In one embodiment, the invention provides a method for predicting the change in the level of at least one pro-inflammatory cytokine in an individual due to alpha tocopherol supplementation, the method comprising analyzing a sample obtained from the human for the presence of one or more genetic variations in at least one gene correlated with a change in serum pro-inflammatory cytokine levels; detecting the genotype of the at least one gene correlated with a with a change in serum pro-inflammatory cytokine levels; and predicting an outcome of the change based on the correlation.

In some embodiments, the at least one pro-inflammatory cytokine is selected from the group consisting of TNFα, IL-6, IL-1, and combinations of the foregoing.

In some embodiments, the at least one gene correlated with a change in serum pro-inflammatory cytokine levels is selected from the group consisting of TNF, IL10, SOD2, GSTP1, and combinations of the foregoing.

In some embodiments, the method comprises:

detecting the genotype of the at least one gene selected from the group consisting of: the TNF genotype at position −308; the TNF genotype at position −238; the IL10 genotype at position −592; the IL10 genotype at position −1082; the IL10 genotype at position −819; the IL10 genotype at position −592; the IL10 genotype at position −1082; the SOD2 genotype at position −28; the GSTP1 genotype at position 313; and combinations of the foregoing; and

predicting an outcome selected from the group consisting of an increase in serum levels of TNFα when IL10-1082 SNP has a genotype of AG; an increase in serum levels of TNFα when IL10-1082 SNP has a genotype of AA; an increase in serum levels of TNFα when the TNF-238 SNP has a genotype of GG; a decrease in serum levels of TNFα when the TNF-238 SNP has a genotype of GA; a decrease in serum levels of IL-6 when the GSTP1 313 SNP has a genotype of GG; an increase in serum levels of IL-6 when the GSTP1 313 SNP has a genotype of AG; an increase in serum levels of IL-6 when the GSTP1 313 SNP has a genotype of GG; an increase in serum levels of IL-6 when the SOD-28 SNP has a genotype of TT; a decrease in serum levels of IL-1 when the GSTP1 313 SNP has a genotype of GG; an increase in serum levels of IL-1 when the IL10-819 SNP has a genotype of CC; and an increase in serum levels of IL-1 when the IL10-1082 SNP has a genotype of GG.

In some embodiments, the level of at least one pro-inflammatory cytokine is a serum level.

In some embodiments, the alpha tocopherol supplementation is about 75 IU/day to about 600 IU/day.

In some embodiments, the alpha tocopherol supplementation is daily for six weeks.

In some embodiments, the gene correlated with a with a change in serum pro-inflammatory cytokine levels is correslated using the false discovery rate (FDR) method.

In some embodiments, the genotpye is determined as part of panel of at least 2 genes that have one or more alleles selected from the group consisting of TNF, IL10, SOD2, GSTP1, and combinations of the foregoing; wherein other genes are selected from methylene-metra-hydro-folate-reductase (MTHFR); methionine synthase reductase (MS-MTRR); methionine synthase (MTR); cystathionine beta synthase (CBS); Manganese superoxide dismutase (MnSOD); superoxide dismutase 3 (SOD3); glutathione S-transferase M1 (GSTM1); glutathione S-transferaseT1 (GSTT1); glutathione S-transferase pi (GSTP1); apolipoprotein C-III (APOC3); apolipoprotein A-V (APOA5); cholesteryl ester transfer protein (CETP); ipoprotein lipase (LPL); endothelial nitric oxide synthase (eNOS); angiotensin converting enzyme gene (ACE); vitamin D receptor (VDR); collagen type 1 alpha 1 (COL1A1); peroxisome proliferator-activated receptor gamma 2 (PPAR-γ2); epoxide hydrolase I (EPHX1); hepatic lipase (LIPC); paraoxonase 1 (PON1); alcohol dehydrogenase IB (ADH1B); alcohol dehydrogenase IC (ADH1C); angiotensinogen (AGT); cytochrome P450 1A1 (CYP1A1); cytochrome P450 1A2*1B (CYP1A2_{—}1B); cytochrome P450 1A2*1E (CYP1A2_{—}1E); and cytochrome P450 1A2*1F (CYP1A2_{—}1F).

In another embodiment, the invention provides a method for predicting the response of an individual to alpha tocopherol supplementation, which method comprises: determining the GSTP1 genotype at position 313; and predicting a greater response to said alpha tocopherol supplementation when the individual is homozygous at the G allele at position 313 than when the individual has another genotype at position 313.

In another embodiment, the invention provides a method of nutrigenetic screening of an individual, the improvement comprising: determining whether the individual has a GG genotype at position 313 of the GSTP1 gene; and; recommending a minimum level of vitamin E intake in the diet to the individual where the GG genotype is present.

### DETAILED DESCRIPTION

The present inventors have identified associations between alleles of certain genes, such as such as GSTP1, SOD2, TNF, and IL10, and changes in at least one pro-inflammatory cytokine, such as IL1, TNF, and IL6 in an individual due to αT supplementation. By assessing whether or not these alleles are present in individuals, it is possible to provide advice on intake and guidance as to αT supplementation. The invention is intended for performance on human subjects. Generally the human will be adult, i.e. age 18 or above. The subjects may be men or women. The associations reported herein were determined in male subjects; however, as none of the genes implicated are sex-linked, no significant differences are predicted to be identified between men and women and thus the associations apply to both sexes. Similarly, it is expected that the invention may be practiced on subjects of any ethnic population group, e.g. caucasians, those of black African or oriental origin, and so forth. As used herein, supplementation can be effected by taking supplements, e.g., αT capsules, or increasing the level of intake of αT or vitamin E by other means, such as dietary changes to increase the amount of foods containing αT or vitamin E ingested. The supplementation may be in any amount. Preferably, the level is any level between about 75 IU/day and about 600 IU/day, inclusive.

The inventors explored the role of functional genetic polymorphisms on serum levels of pro-inflammatory cytokines in response to α-tocopherol supplementation. 159 healthy middle-aged male volunteers (mean age 52.7 years) were given dietary supplements of either 75 IU (moderate dose n=57), or 600 IU (high dose n=102) RRR-α tocopherol (αT) per day. Peripheral blood mononuclear cell (PBMC) levels of circulating TNF-α, interleukin-1 (IL-1) and interleukin-6 (IL-6) were measured at baseline and after six weeks. Genotypes at 21 polymorphisms in 15 genes involved in anti-oxidant response or inflammation (table 1) were determined.

The change in TNF-α PBMC levels as a result of αT supplementation was affected by polymorphism TNFa-238 (p<0.001) and by IL10 1092 (p<0.02). Change in IL-6 levels was affected by the GSTP1 31 variant (p<0.001) Three IL10 SNPs were associated with change in IL-1 levels (p<0.0004). These associations remained statistically significant after adjustment for multiple comparisons. Among healthy controls the inflammatory response to αT supplementation appears to be strongly dependent on an individual's genotype. These differences may help explain some of the discrepant results from vitamin E observational and interventional studies.

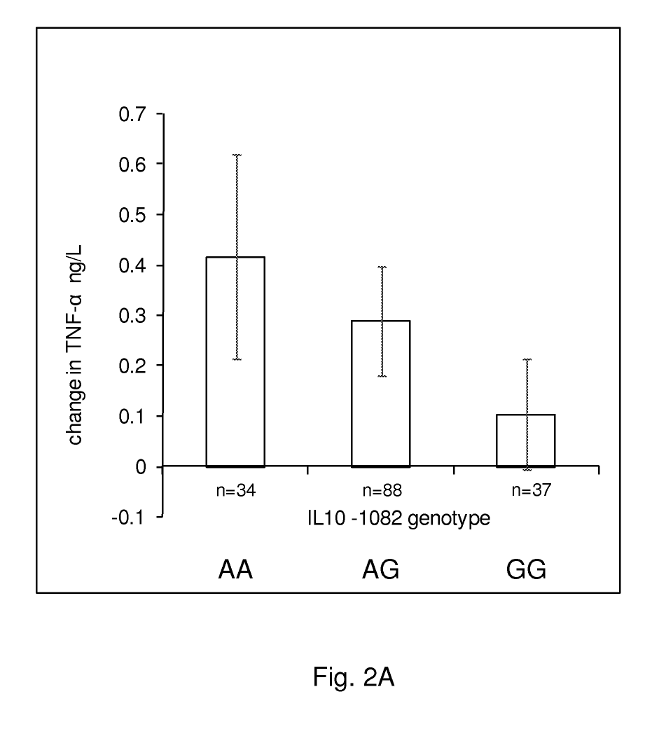

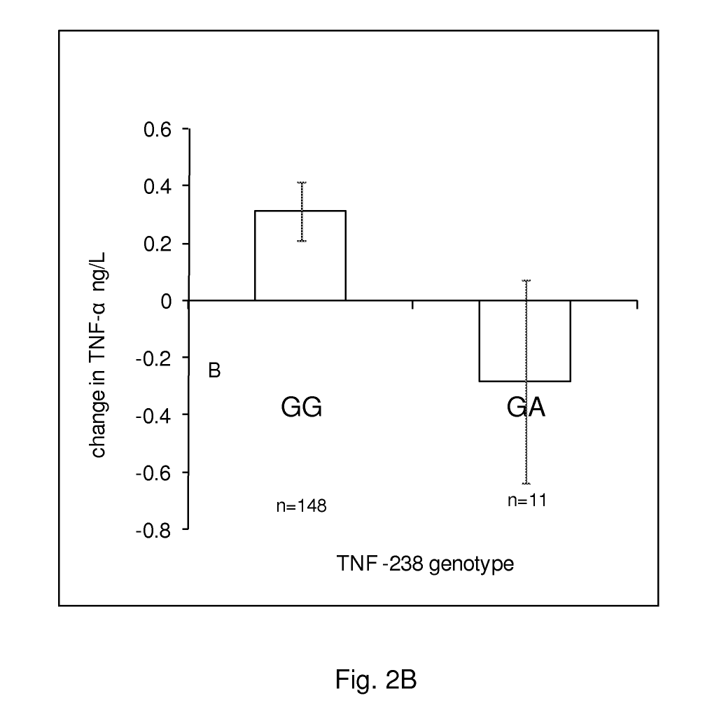

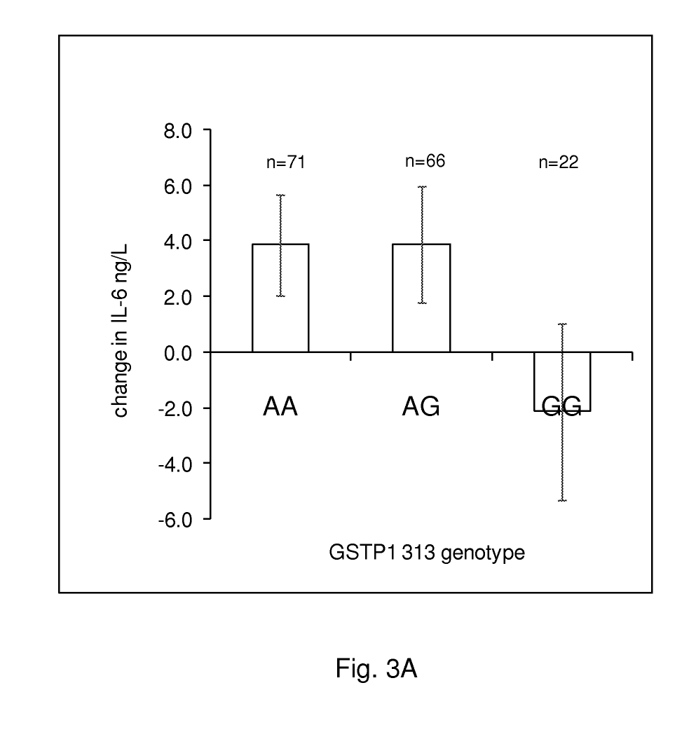

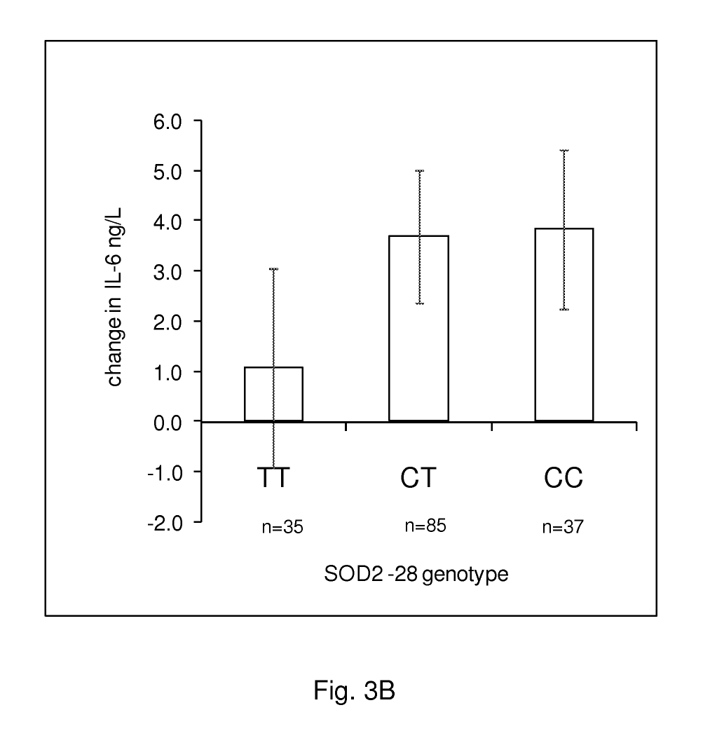

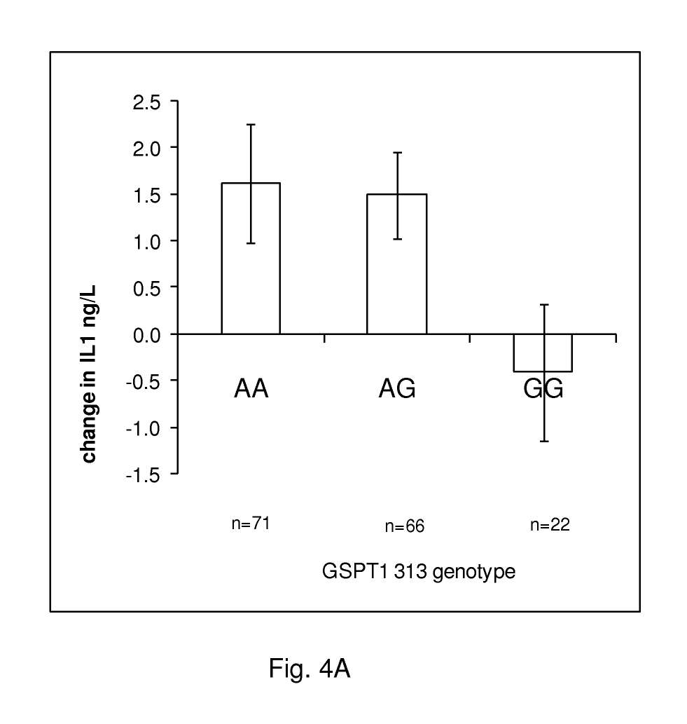

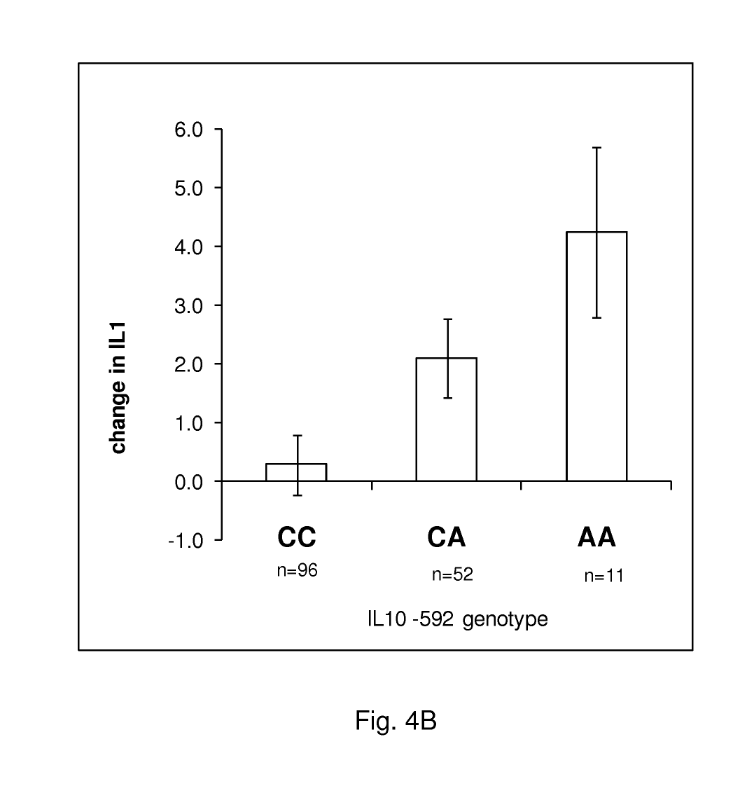

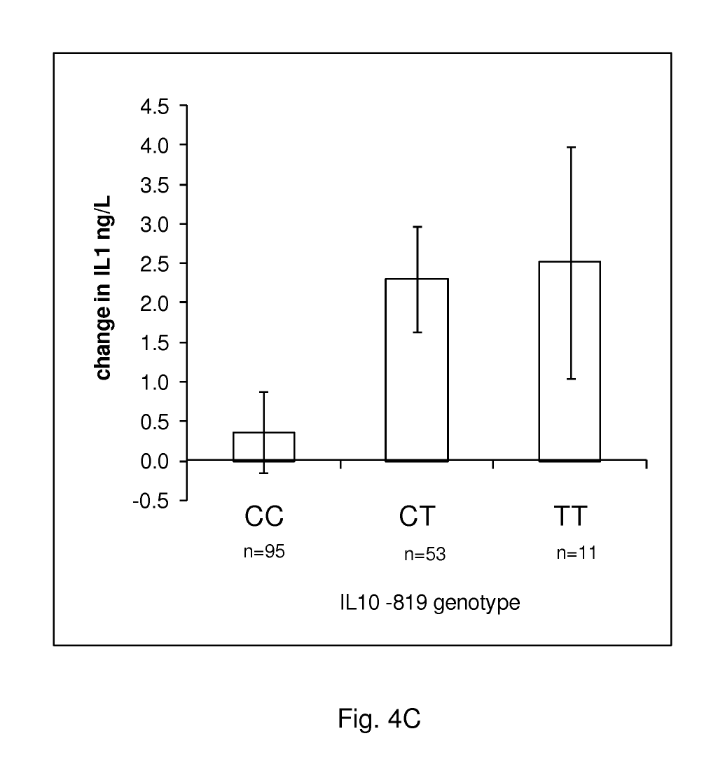

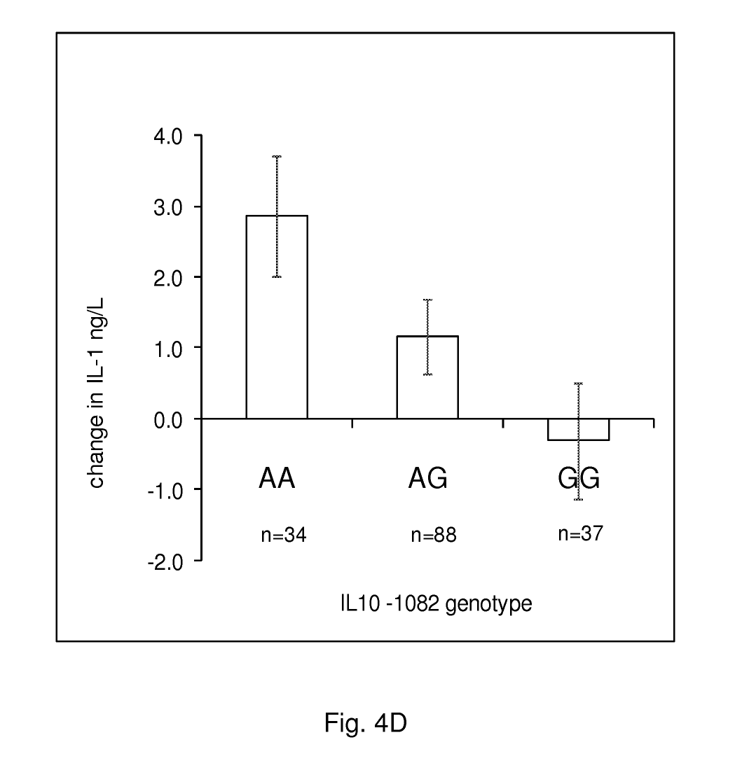

More particularly, in this study the inventors found that several polymorphisms were found to be associated with change in cytokine serum levels (table 3). Genotypes AG and AA for the IL10-1082 SNP and genotype GG for the TNF-238 SNP were associated with an increase in serum levels of TNFα (FIG. 2). The decrease in TNFα in response to αT supplementation for the less common GA genotype at TNF-238 remained statistically significant (p<0.016) after correcting for multiple tests. The variant homozygotes at the GSTP1 313 (GG) polymorphism was significantly associated with decreased levels of IL-6 whereas the AG and GG carriers showed an increase in IL-6 serum levels. The SOD-28 TT polymorphisms was associated with a lower increase in IL-6 levels than the CT and CC variants (FIG. 3B). The GG genotype at the GSTP1 313 SNP was also associated with a decrease in IL-1 levels (FIG. 4A) although the difference was not statistically significant after the FDR correction. All three IL10 variants tested were associated with differences in the change of IL1. The common genotypes at IL10-592 (CC) and at IL10-819 were both associated with a lower increase in IL1 levels. (FIG. 4B and FIG. 4C). The GG carriers at the IL10-1082 also showed a statistically significant lower increase in IL1 levels (FIG. 4D). These differences remained statistically significant or nearly so after adjustment for multiple tests.

Individuals with the GG genotype at GSTP1 313 had higher levels of IL-6 than did AA or AG carriers although this difference did not achieve statistical significance. Similarly carriers of the IL10-1082 GG genotype had not significantly higher levels of IL-1 at baseline than AA or AG carriers. Finally, carriers of the variant AG genotype at TNF-238 had significantly higher levels of TNFα than the GG homozygotes.

The GG genotype at GSTP1 313 has been implicated in a worse response to oxidative stress, e.g. response to ozone exposure (22), or to smoking (23). In this invention, in all three cases the genotype associated with a lower inflammatory response to αT supplementation was the one that was associated with higher inflammation at baseline. Because all the analyses were adjusted for baseline values, the data suggest that the individuals who benefit the most from antioxidant therapy are those who were in a worse position at baseline. Subjects with lower antioxidant capacity due to their genetic background derive a greater advantage from α T supplementation. This would appear consistent with what has been observed in clinical trials, where individuals with impaired antioxidant capacity have been reported to draw the greatest health benefit in terms of decreased risk of CVD from vitamin E (10). The data suggest that individuals with worse anti-oxidant or anti-inflammatory capacity will be the ones benefiting the most from intake of αT.

The data of the present invention show that in subjects with a certain alleles, the response to αT supplementation was greater than in subjects with wild-type alleles. Thus, the polymorphisms of the present invention may be used by individuals or heath care practitioners to predict responses to αT supplementation.

Accordingly, the findings of the present invention may be used to provide both general dietary and/or lifestyle advice based on the GSTP1, SOD2, TNF, and/or IL10 genotypes of individuals, as well as more specific advice on food subtype intake, i.e. in regard to one or more of vitamin E, αT, and foods containing vitamin E or αT. This information may be used to advise individuals that their response to αT supplementation may be different than those with other alleles. The exact degree of response will depend on the nature of the diet and/or supplementation regime to accompany the diet.

### GSTP1 Genotype

The glutathione S-transferase pi gene (GSTP1) is a polymorphic gene encoding active, functionally different GSTP1 variant proteins that are thought to function in xenobiotic metabolism and play a role in susceptibility to cancer, and other diseases. However, no associations with normal dietary factors have been reported to date. There are two common allelic variants of GSTP1. One is at position 313 of the open reading frame of the nucleic acid, which changes A to G, the other at position 341 is a C to T change. Both changes also cause a change to the coding sequence, resulting in the protein variants Ile105Val and Ala114Val. The gene is autosomal; thus individuals can be homozygous or heterozygous at each allele.

The sequence of the GSTP1 gene and translation thereof is shown as SEQ ID NO:7 (Accession number: NG_{—}012075.1 GI:237820690) and SEQ ID NO:8 (Accession number NP_{—}000843.1 GI:4504183) respectively. The wild-type sequence is shown. As indicated above, the changes at positions 313 and 341 also give rise to coding sequences and are also referred to in the literature as Ile105Val and Ala114Val respectively. In this invention we refer to the alleles by reference to the nucleotide numbering and changes, since genetic screening is primarily done by reference to nucleotide analysis.

Single nucleotide polymorphisms are also classified by the Database of Single Nucleotide Polymorphisms (dbSNP), Bethesda (MD): National Center for Biotechnology Information, National Library of Medicine (see Sherry S T, et al; dbSNP: the NCBI database of genetic variation. Nucleic Acids Res. 2001 Jan. 1; 29(1):308-11). The SNPs are catalogued by unique accession numbers. In the present case, the GSTP1 A313G polymorphism is SNP accession number rs1695.

### TNF Genotype

The tumor necrosis factor gene (TNF) is a polymorphic gene that regulates expression of several receptors in vascular endothelial cells and exerts a variety of physiological and pathogenic effects that lead to tissue destruction. TNF promoter polymorphisms affect transcriptional activation. No known associations with normal dietary factors have been reported to date. There are a number of common allelic variants of TNF. One is at position −308 of the open reading frame of the nucleic acid, which changes G to A, another at position −238 is a G to A change. The gene is autosomal; thus individuals can be homozygous or heterozygous at each allele.

The sequence of the TNF gene and translation thereof is shown as SEQ ID NO:1 (Accession number NG_{—}007462.1 GI:170014716) and SEQ ID NO:2 (Accession number NP_{—}000585.2 GI:25952111) respectively. The wild-type sequence is shown.

### IL10 Genotype

The interleukin 10 gene (IL10) is a polymorphic gene encoding is a regulatory cytokine that limits inflammatory processes. The quantitative production of IL-10 is subject to genetic variation based on polymorphisms in the promoter of its gene. However, no known associations with normal dietary factors have been reported to date. The gene is autosomal; thus individuals can be homozygous or heterozygous at each allele.

The sequence of the IL10 gene and translation thereof is shown as SEQ ID NO:3 (Accession number: NG_{—}012088.1 GI:237858687) and SEQ ID NO:4 (Accession number NP_{—}000563.1 GI:10835141) respectively. The wild-type sequence is shown.

### SOD2 Genotype

The superoxide dismutase 2 gene (SOD2) is a polymorphic gene encoding a protein having a major role in protecting the mitochondrion from oxidative damage due to superoxide radicals and other excited oxygen species. The quantitative production of IL-10 is subject to genetic variation based on polymorphisms in the promoter of its gene. However, no known associations with normal dietary factors have been reported to date. The gene is autosomal; thus individuals can be homozygous or heterozygous at each allele.

The sequence of the SOD2 gene and translation thereof is shown as SEQ ID NO:5 (Accession number: NG_{—}008729.1 GI:209447079) and SEQ ID NO:6 (Accession number NP_{—}001019636.1 GI:67782307) respectively. The wild-type sequence is shown.

Where available, the identification number from the “National Center for Biotechnology Information's (NCBI's) reference sequence database (Accession # and gi #) and additional information (e.g., the name or sequence of the peptide marker as contained in the NCBI queried database is also provided. An accession number is the unique identifier for a sequence record. An accession number applies to the complete record. Accession numbers do not change, even if information in the record is changed at the author's request. A gi number or “GenInfo Identifier” sequence identification number refers to a sequence identifier that runs parallel to the accession.version system. Therefore, if the sequence changes in any way, it will receive a new gi number. Thus, the combination of accession number and gi number provides a clear and unambiguous source of a given biosequence as of the filing date.

The genotype of an individual will generally be determined by analysis of a sample of nucleic acid, normally DNA, obtained from the individual, e.g. in the form of a buccal swab or similar sample. The analysis will take place using conventional methods known as such in the art. This may include use of PCR to amplify and sequence the gene at one or both positions 313 and 341 or the use of nucleic acid probes that are capable of distinguishing between the alleles by differentially hybridizing to the wild-type and variant sequences.

In many embodiments of the invention, the alleles of genes correlated with a change in pro-inflammatory cytokine levels, such as GSTP1, SOD2, TNF, and/or IL10, will be determined in a gene chip array in which a number of other gene variants associated with lifestyle and dietary risk factors are also analysed.

Since some of the alleles are also reflected in protein coding changes it is possible that the alleles may be detected at the protein level, e.g. by immunoassay or other protein analytical methods. Such methods would be practiced using a sample from the individual which contains detectable levels of expressed protein corresponding to the above-mentioned genes.

As well as predicting the relative likely cytokine response to αT, the invention may also allow the opportunity to tailor dietary or supplementation programs based on the genotypes disclosed herein. For example, individuals with a GG genotype at GSTP1 313, could be counselled not only in their expectations of likely inflammatory response to αT supplemention, but also given guidance and advice on means to achieve greater response by either modifying the level of αT supplementation (e.g., to 75 IU/day or 600 IU/day), or engaging in αT supplementation for a different period of time (e.g., up to six weeks).

The field of nutrigenetic screening involves the analysis of one or more genes in a subject which is involved in a response to a dietary or other health-associated factor, and in which one or more alleles that may alter that response have been identified.

Various methods of determining a personalised lifestyle advice plan for human subjects are disclosed in U.S. Pat. No. 7,054,758, the disclosure of which is incorporated herein by reference. In general, methods comprise the steps, which are usually computer-assisted, of:
(i) providing a first dataset on a data processing device, said first dataset comprising information correlating the presence of individual alleles known to be associated with increased or decreased disease susceptibility, with a lifestyle risk factor;
(ii) providing a second dataset on a data processing device, said second dataset comprising information matching each said risk factor with at least one lifestyle recommendation;
(iii) inputting a third dataset identifying alleles present in said subject, wherein said alleles are one or more of the alleles of said first dataset;
(iv) determining the risk factors associated with said alleles present in said human subject by correlating said alleles with risk factors provided by said first dataset;
(v) determining at least one lifestyle recommendation based on each identified risk factor from step (iv) by matching said risk factor with a lifestyle recommendation from said second dataset; and
(vi) generating a personalized lifestyle advice plan based comprising at least one lifestyle recommendation determined in step (v).

The personalized lifestyle advice plan may include recommended minimum and/or maximum amounts of food subtypes. The associations disclosed herein with the genes correlated with a change in pro-inflammatory cytokine levels, such as GSTP1, SOD2, TNF, and/or IL10 genes may be used to provide alleles for step (i) of the above process, risk factors relating to step (ii) and the recommendations disclosed herein for step (v) in order to generate a personalised lifestyle advice plan which takes account of the genotype of genes correlated with a change in pro-inflammatory cytokine levels, such as, GSTP1, SOD2, TNF, and/or IL 10, amongst other genetic markers.

In a typical procedure, a sample of DNA from a subject is provided. This may be in the form of a buccal swab or other body sample. The DNA is then examined to determine which alleles of one or more genes of interest are present. Where alleles of genes which are identified that give rise to increased risk of one or more adverse outcomes (e.g. lower bone mineral density, higher risk of heart disease, etc) the individual may be advised to modify his or her diet by to account for that risk. For example, the advice may include recommended minimum and/or maximum amounts of food subtypes, such as fats, vegetable subgroups (brassicas, alliums, etc). Such a method may be the method of U.S. Pat. No. 7,054,758 referred to above.

In some embodiments of nutrigenetic screening, the individual may also provide, in conjunction with a DNA sample, a response to a questionnaire providing lifestyle details (for example such as one or more of current diet, age, sex, alcohol intake and whether or not they are a smoker). This can allow the advice to be further tailored to the requirements of the individual.

In a typical method of nutrigenetic screening, the alleles of GSTP1, SOD2, TNF, and/or IL10 may be determined within a panel of from 2 to 100, such as from 2 to 20 or 5 to 20 other genes which have allelic variants associated with responses to, or risk factors for, diet or health. The genes which may be included in the panel may be selected from methylene-metra-hydro-folate-reductase (MTHFR); methionine synthase reductase (MS-MTRR); methionine synthase (MTR); cystathionine beta synthase (CBS); Manganese superoxide dismutase (MnSOD); superoxide dismutase 3 (SOD3); glutathione S-transferase M1 (GSTM1); glutathione 5-transferaseT1 (GSTT1); interleukin-6 (IL-6); apolipoprotein A-V (APOA5); apolipoprotein C-III (APOC3); cholesteryl ester transfer protein (CETP); lipoprotein lipase (LPL); endothelial nitric oxide synthase (eNOS); angiotensin converting enzyme gene (ACE); vitamin D receptor (VDR); collagen type 1 alpha 1 (COL1A1); peroxisome proliferator-activated receptor gamma 2 (PPAR-γ2); epoxide hydrolase I (EPHX1); hepatic lipase (LIPC); paraoxonase 1 (PON1); alcohol dehydrogenase IB (ADH1B); alcohol dehydrogenase IC (ADH1C); angiotensinogen (AGT); cytochrome P450 1A1 (CYP1A1); cytochrome P450 1A2*1B (CYP1A2_{—}1B); cytochrome P450 1A2*1E (CYP1A2_{—}1E); and cytochrome P450 1A2*1F (CYP1A2_{—}1F).

The polymorphisms of the gene panel for the above genes, when included in the panel, may be selected from the following:

TABLE 4

| | Genetic | rs Number |
|---|---|---|
| Gene | Variations | (where applicable) |
| MTHFR | C677T; | rs1801133 |
| | A1298C | rs1801131 |
| MS-MTRR | A66G | rs1801394 |
| MTR | A2756G | rs1805087 |
| CBS | C699T | rs234706 |
| MnSOD | C(−28)T | rs4880 |
| SOD3 | C760G | rs1799895 |
| GSTM1 | Present or Deleted | n/a |
| GSTT1 | Present or Deleted | n/a |
| APOA5 | T1131C; | rs662799 |
| | C56G | rs3135506 |
| IL-6 | G(−174)C; | rs1695 |
| | G(−634)C | rs1138272 |
| APOC3 | C3175G | rs5128 |
| CETP | G279A | rs708272 |
| LPL | C1595G | rs328 |
| eNOS | G894T | rs1799983 |
| ACE | Insertion/Deletion | rs4646994 |
| VDR | TBsmIC; | rs1544410 |
| | CTaqIT; | rs731236 |
| | TFokIC | rs10735810 |
| COL1A1 | GSp1T | rs1800012 |
| PPAR-γ2 | ProI2 AIA | rs1801282 |
| EPHX1 | Tyr113His | rs1051740 |
| LIPC | G250A; | rs2070895 |
| | C514T | rs1800588 |
| PON1 | Gln192Arg; | rs662 |
| | Leu55Met | rs854560 |
| ADH1B | Arg369Cys; | rs2066702 |
| | Arg47His | rs1229984 |
| ADH1C | Ile349Val | rs698 |
| AGT | Met235Thr | rs699 |
| CYP1A1 | A2455G | rs1048943 |
| CYP1A2_1B | C 1548T | rs2470890 |
| CYP1A2_lE | G740GT | rs2069526 |
| CYP1A2_1F | A163C | rs762551 |

Thus all the methods of the present invention described herein may be practiced either on the TNF, IL10, SOD2, and GSTP1 genes alone, or in various combinations as part of a nutrigenetic screening method. When the latter, the method may include the determination of an allele of one or more of the genes of the above Table 4.

### Example 1

In this study, the inventors selected 21 variants in 15 genes known to influence an individual's inflammatory or oxidative stress response (table 1) and assessed the role that genotypes at these genes had on serum cytokine levels among healthy men after 6 weeks of αT supplementation.

### Subjects and Methods

Healthy middle-aged male volunteers (mean age 52.7±10 years; median years) were given dietary supplements of either 75 IU (moderate dose), or 600 IU (high dose) RRR-α tocopherol per day for six weeks. Subject numbers in each group were 57 and 102 respectively. All research participants gave written informed consent to take part. The study protocol was approved by the Southampton University Hospitals Trust Clinical Ethics Committee authorized the research protocol. Study participants continued with their normal diets while receiving the supplements. Subjects continued with their normal daily activities while on the study but visited the Wellcome Trust Clinical Research Facility at Southampton University Hospital Trust, after an overnight fast, at the beginning and end of the supplementation period. At the visit weight, height, and waist and hip circumference were measured, and blood samples taken for the measurement of cytokines.

Alpha-tocopherol in plasma was determined using fluorimetry according to methods described elsewhere (11). The limit of detection for αT was 7.2 μmol/L. Peripheral blood mononuclear cells (PBMCs) were prepared from heparinized whole blood samples using Leucosep sterile centrifuge tubes (Greiner Bio-one, Germany) and Histopaque gradient solution (Sigma-Aldrich Inc, MO, USA) and cultured in the presence of Lipopolysaccharide (LPS) (Sigma-Aldrich Inc, MO, USA) for 24 h after which time the supernatants were removed and stored. Interleukin-6 (IL-6), tumor necrosis factor (TNF) and interleukin-1β (IL-1β) protein levels were quantitatively measured by the BD CBA Human Inflammation Kit (BD Bioscience, San Jose, Calif., USA). The operations were performed according to the manufacturer's instructions. The intensity of the fluorescence signal was acquired on a fluorescence activated cell sorter (FACS) flow cytometer (BD Becton-Dickinson), and analyzed using CBA software. The concentration range for detection using this assay is 20-5000 pg/ml for each of the proteins.

### Genotyping:

DNA was extracted from EDTA anticoagulated blood using a salting out method described previously (12). Briefly, cell membranes were lysed using detergent, proteins digested using proteinase K (Sigma-Aldrich Inc, MO, USA) and removed in 6 M sodium chloride (Sigma-Aldrich Inc, MO, USA) with centrifugation. DNA was precipitated from the remaining solution in ethanol, dried and dissolved in distilled water. Genotyping was performed using the Taqman ABI 7700 sequence detection system (Applied Biosystems, Foster City, Calif., USA). The list of genetic variants tested in genes TNF, LTA, IL10, SLC11A1, IL6, NOS3, IL1A, IL1B, 1L4, PPARG, MTHFR, SOD2, GSTM1, GSTT1, and GSTP1 is listed in table 1. With the exception of the null GSTM1 and GSTT1 variants for which only two genotype states are possible (present or null) all genotypes were in Hardy Weinberg equilibrium (table 1). Single nucleotide polymorphisms are also classified by the Database of Single Nucleotide Polymorphisms (dbSNP), Bethesda (MD): National Center for Biotechnology Information, National Library of Medicine (see Sherry S T, et al; dbSNP: the NCBI database of genetic variation. Nucleic Acids Res. 2001 Jan. 1; 29(1):308-11). The SNPs are catalogued by unique accession numbers. In the present case, the GSTP1 A313G polymorphism is SNP accession number rs1695. The sequence of the GSTP1 gene (cDNA) and translation thereof is shown as SEQ ID NO:1 and SEQ ID NO:2 respectively. The wild-type sequence is shown. The numbering is based on the open reading frame, with the first methionine ATG being numbered 1-3 of the sequence. The change at position 313 gives rise to coding sequences and is also referred to in the literature as Ile105Val.

### Statistical Methods

Genetic Association Analysis: A co-dominant (additive) genetic model was tested, which assumes that the mean value of a trait (in this case change in cytokine serum levels) for the heterozygote is between that of the to homozygotes. The genotype was thus coded as 0=major allele homozygote 1=heterozygote 2=minor allele homozygote.

A linear regression was carried out where the outcome variable was Log(DeltaCk+minDeltaCk) Where Ck stands for IL-1, IL-6 or TNF-αlpha. And DeltaCk=Followup. Baseline serum cytokine levels, and minDelta the most negative DeltaCk (<0) observed in the population baseline value, alpha tocopherol dose group, smoking status (age+BMI) were included as covariates.

Adjustment for multiple comparisons: The false discovery rate probability method (FDR) (13) was used to adjust for multiple testing.

### Results:

The descriptive statistics of study subjects are shown in Table 2. The serum levels of three pro-inflammatory cytokines interleukin 6 (IL-6), Interleukin 1 beta (IL-1) and tumor necrosis factor 1 alpha (TNFα) were used as outcome variables. The mean levels of these three cytokines before and after supplementation in the two study groups (75 UI and 600 UI) are shown in table 2. Overall, no significant differences in age, smoking status BMI or any of the three cytokines were observed between the two treatment groups. An increase in plasma fasting levels of alpha-tocopherol was seen in both dose groups, although the increase was much larger (Table 2).

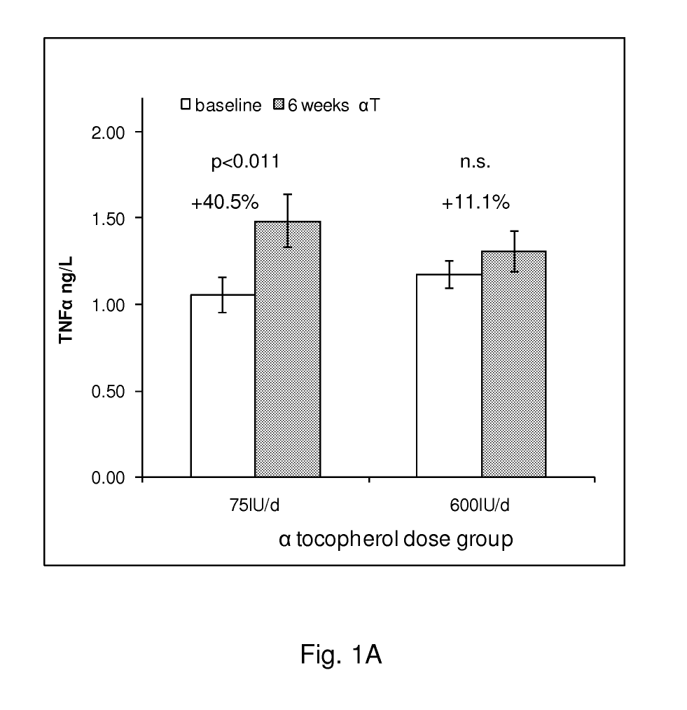

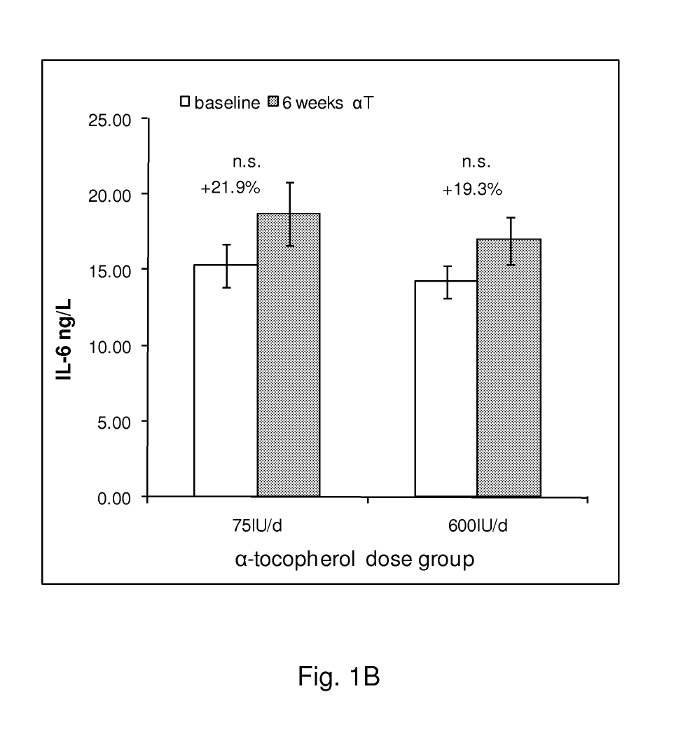

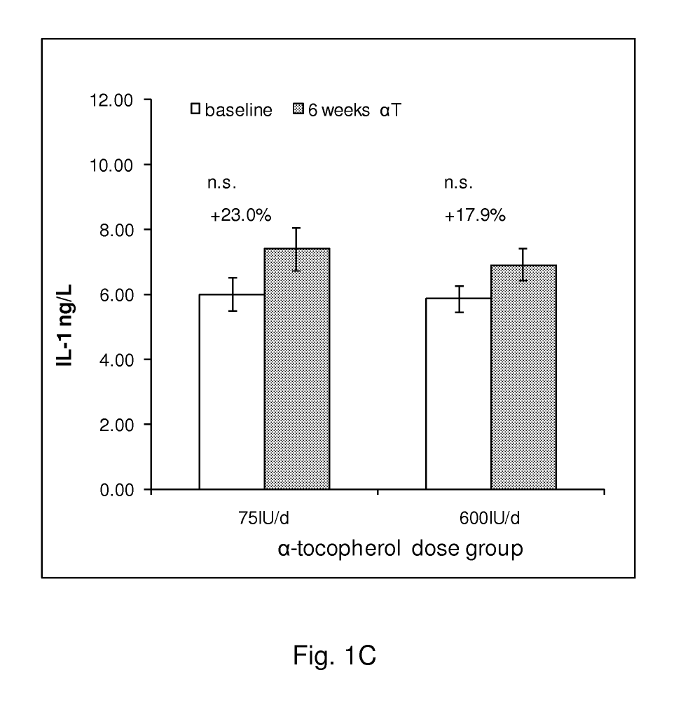

The mean change in cytokine levels are shown in FIG. 1A-1D. An increase in the serum levels of all three cytokines was observed in both treatment groups. For TNFα in the low-dose group a 40.5% increase was observed after supplementation which was statistically significant (p<0.011) after adjusting for age, baseline levels, smoking status and BMI.

The change in cytokine serum levels after 6 weeks of αT supplementation was then compared between genotypes, adjusting for baseline cytokine levels, age, BMI, smoking status and αT dose. Before adjusting for multiple tests several polymorphisms were found to be associated with change in cytokine serum levels (table 3). Genotypes AG and AA for the IL10-1082 SNP and genotype GG for the TNF-238 SNP were associated with an increase in serum levels of TNFα (FIG. 2). The decrease in TNFα in response to αT supplementation for the less common GA genotype at TNF-238 remained statistically significant (p<0.016) after correcting for multiple tests.

The variant homozygotes at the GSTP1 313 (GG) polymorphism was significantly associated with decreased levels of IL-6 whereas the AG and GG carriers showed an increase in IL-6 serum levels (p<0.019, FIG. 3A, Table 3). The SOD-28 TT polymorphisms was associated with a lower increase in IL-6 levels than the CT and CC variants (FIG. 3B), but this association was not significant after adjusting for multiple tests.

The GG genotype at the GSTP1 313 SNP was also associated with a decrease in IL-1 levels (FIG. 4A) although the difference was not statistically significant after the FDR correction. All three IL10 variants tested were associated with differences in the change of IL1. The common genotypes at IL10-592 (CC) and at IL10-819 were both associated with a lower increase in IL1 levels. (FIG. 4B and FIG. 4C). The GG carriers at the IL10-1082 also showed a statistically significant lower increase in IL1 levels (FIG. 4D). These differences remained statistically significant or nearly so after adjustment for multiple tests (p<0.025, p<0.053 and p<0.016 respectively).

Individuals with the GG genotype at GSTP1 313 had higher levels of IL-6 (14.95±1.05) than did AA or AG carriers (14.52±1.14 ng/L) although this difference did not achieve statistical significance. Similarly carriers of the IL10-1082 GG genotype had not significantly higher levels of IL-1 at baseline (6.75±1.15 ng/L) than AA or AG carriers (5.87±0.33 ng/L). Finally, carriers of the variant AG genotype at TNF-238 had significantly higher (p<0.011) levels of TNFα (1.66±0.22) than the GG homozygotes (1.07±0.06).

The disclosure of U.S. Patent Application Ser. No. 61/153,606 is hereby incorporated by reference.

### REFERENCES

1. Singh U, Jialal I. Anti-inflammatory effects of alpha-tocopherol. Ann N Y Acad Sci. 2004; 1031:195-203.
2. Singh U, Devaraj S, Jialal I. Vitamin E, oxidative stress, and inflammation Annu Rev Nutr. 2005; 25:151-74.
3. Thomas S R, Stocker R. Molecular action of vitamin E in lipoprotein oxidation: implications for atherosclerosis. Free Radic. Biol. Med. 2000; 28(12):1795-805
4. Kaul N, Devaraj S, Jialal I. Alphatocopherol and atherosclerosis. Exp. Biol. Med. 2001: 226:5-125.
5. Schock B C, Van der Vliet A, Corbacho A M, et al. Enhanced inflammatory responses in alpha-tocopherol transfer protein null mice. Arch. Biochem. Biophys. 2004; 423:162-69
6. Stampfer M J, Hennekens C H, Manson J E, Colditz G A, Rosner B, Willett W C. Vitamin E consumption and the risk of coronary disease in women. N Engl J. Med. 1993; 328(20): 1444-9.
7. Knekt P, Reunanen A, Järvinen R, Seppänen R, Heliövaara M, Aromaa A. Antioxidant vitamin intake and coronary mortality in a longitudinal population study. Am J Epidemiol. 1994; 139(12):1180-9.
8. Muntwyler J, Hennekens C H, Manson J E, Buring J E, Gaziano J M. Vitamin supplement use in a low-risk population of US male physicians and subsequent cardiovascular mortality. Arch Intern Med. 2002; 162(13):1472-6
9. Thomson M J, Puntmann V, Kaski J C. Atherosclerosis and oxidant stress: the end of the road for antioxidant vitamin treatment? Cardiovasc Drugs Ther. 2007; 21(3):195-210.
10. Hodis F I N, Mack W J, LaBree L, et al. Alpha-tocopherol supplementation in healthy individuals reduces low-density lipoprotein oxidation but not atherosclerosis: the Vitamin E Atherosclerosis Prevention Study (VEAPS). Circulation. 2002 Sep. 17; 106(12):1453-9.
11. Driskell W J, Neese J W, Bryant C C, Bashor M M. Measurement of vitamin A and vitamin E in human serum by high-performance liquid chromatography. J. Chromatogr. 1982; 10; 231(2):439-44.
12. Miller S A, Dykes D D, Polesky H F. A simple salting out procedure for extracting DNA from human nucleated cells. Nucleic Acids Res. 1988 Feb. 11; 16(3):1215
13. Benjamini Y, Hochberg Y. Controlling the False Discovery Rate—a new and powerful approach to multiple testing. J Royal Stat Soc Ser. 1995; B 57:289-300.
14. Cuschieri J, Maier R V. Oxidative stress, lipid rafts, and macrophage reprogramming. Antioxid Redox Signal. 2007 September; 9(9):1485-97.
15. Yusuf S. Clinical, public health, and research implications of the Heart Outcomes Prevention Evaluation (HOPE) Study. Eur Heart J 2001; 22: 103-4.
16. MRC/BHF Heart Protection Study of antioxidant vitamin supplementation in 20,536 high-risk individuals: a randomised placebo-controlled trial. Lancet 2002; 360:23-33.
17. Vivekananthan D P, Penn M S, Sapp S K, Hsu A, Topol E J. Use of antioxidant vitamins for the prevention of cardiovascular disease: metaanalysis of randomised trials. Lancet 2003; 361:2017-23.
18. Boaz M, Smetana S, Weinstein T, et al. Secondary prevention with antioxidants of cardiovascular disease in endstage renal disease (SPACE): randomised placebo-controlled trial. Lancet. 2000 Oct. 7; 356(9237):1213-8.
19. Salonen R M, Nyyssonen K, Kaikkonen J, et al. Six-year effect of combined vitamin C and E supplementation on atherosclerotic progression: the Antioxidant Supplementation in Atherosclerosis Prevention (ASAP) Study. Circulation 2003; 107:947-53.
20. Fang J C, Kinlay S, Beltrame J, et al. Effect of vitamins C and E on progression of transplant-associated arteriosclerosis: a randomised trial. Lancet 2002; 359:1108-13.
21 Wu J H, Ward N C, Indrawan A P, Almeida C A, Hodgson J M, Proudfoot J M, Puddey I B, Croft K D. Effects of alpha-tocopherol and mixed tocopherol supplementation on markers of oxidative stress and inflammation in type 2 diabetes. Clin Chem. 2007 March; 53(3):511-9.
22. Romieu I, Ramirez-Aguilar M, Sienra-Monge J J, et al. GSTM1 and GSTP1 and respiratory health in asthmatic children exposed to ozone. Eur Respir J. 2006 November; 28(5):953-9
23. Kim J H, Park S G, Lee K H, Choi J H, Ha E H, Myung S K, Hong Y C. GSTM1 and GSTP1 polymorphisms as potential factors for modifying the effect of smoking on inflammatory response. J Korean Med Sci. 2006 December; 21(6):1021-7.

TABLE 1
Genes and genetic polymorphisms studied.

| official gene | | | | | ref SNP | | |
|---|---|---|---|---|---|---|---|
| symbol | chr | Gene | polymorphism | Description | ID⁽¹⁾ | MAF⁽²⁾ | HWE⁽³⁾ |
| TNF | 6 | tumor necrosis factor | TNF - 308 | −308 G > A | rs1800629 | 15.41% | 0.40 |
| | | | TNF -238 | −238 G > A | rs361525 | 3.46% | 0.90 |
| LTA | 6 | lymphotoxin alpha | LTA | intron 1 252 A > G | rs909253 | 32.70% | 1.00 |
| IL10 | 1 | interleukin 10 | IL10 1082 | −1082 A > G | rs1800896 | 49.06% | 0.40 |
| | | | IL10 819 | −819 C > T | rs1800871 | 23.58% | 0.64 |
| | | | IL10 592 | −592 C > A | rs1800872 | 23.27% | 0.57 |
| SLC11A1 | 2 | natural resistance-associated macrophage | NRAMP vntr | VNTR | N/A | 6.88% | 0.96 |
| | | protein 1 | | microsatellite | | | |
| IL6 | 7 | interleukin 6 | IL6 −174 | −174 G > C | rs1800795 | 42.14% | 0.97 |
| | | | IL6 −634 | −634 G > C | rs1800796 | 5.66% | 0.77 |
| NOS3 | 7 | nitric oxide synthase 3 | eNOS 894 | 894 G > T | rs1799983 | 38.99% | 0.96 |
| IL1A | 2 | interleukin 1 alpha | IL1A-889 | −889 C > T | rs1800587 | 27.99% | 0.98 |
| IL1B | 2 | interleukin 1 beta | ILB -511 | −511 C > T | rs16944 | 34.28% | 0.84 |
| IL4 | 5 | interleukin 4 | IL4 vntr | VNTR | N/A | 37.41% | 0.48 |
| | | | | microsatellite | | | |
| PPARG | 3 | peroxisome proliferator-activated | PPARG 12 | 12 Pro > Ala | rs1801282 | 12.66% | 0.94 |
| | | receptor | | | | | |
| | | gamma | | | | | |
| MTHFR | 1 | methylenetetrahydrofolate reductase | MTHFR 677 | 677 C > T | rs1801133 | 27.36% | 0.94 |
| | | intermediate form | MTHFR 1298 | 1298 A > C | rs1801131 | 35.22% | 0.73 |
| SOD2 | 6 | manganese superoxide dismutase | SOD2 −28 | −28 C > T | rs4880 | 49.37% | 0.49 |
| GSTP1 | 11 | glutathione S-transferase pi | GSTP1 313 | 313 A > G | rs1695 | 34.59% | 0.58 |
| | | | GSTP1 341 | 341 C > T | rs1138272 | 8.75% | 0.74 |
| GSTM1 | 1 | glutathione S-transferase M1 | GSTM1 Null | Deletion | N/A | 44.03% | N/A |
| GSTT1 | 22 | glutathione S-transferase theta 1 | GSTT1 Null | Deletion | N/A | 19.50% | N/A |

⁽¹⁾Database of Single Nucleotide Polymorphisms (dbSNP). Bethesda (MD): National Center for Biotechnology Information, National Library of Medicine. dbSNP accession: {ss1 or ss1-ss100}, (dbSNP Build ID: 129). Available from: the NCBI Entrez SNP website.
⁽²⁾Minor allele frequency
⁽³⁾ Test for Hardy Weinberg Equilibrium

TABLE 2
Descriptive characteristics of study subjects

| | | Study Group (αT dose) | | | | |
|---|---|---|---|---|---|---|
| Trait | | 75 IU/d | | 600 IU/d | | p-value |
| Sample Size | | n = 57 | | n = 102 | | |
| smokers % | | 7.00% | | 6.80% | | 0.90 |
| Sex M % | | 100% | | 100% | | |
| age years mean (SD) | | 53.79 | 10.19 | 52.08 | 9.98 | 0.30 |
| BMI kg/m² mean (SD) | | 26.65 | 4.35 | 25.74 | 3.32 | 0.14 |
| fasting plasma αT | baseline | 16.5 | (15.1-17.9) | 18.3 | (17.1-19.6) | 0.08 |
| μmol/L mean (95% CI) | 6-weeks | 22.6 | (20.5-24.6) | 31.3 | (1.16-1.46) | 9 × 10⁻⁹ |
| PMBC cytokine levels | | | | | | |
| TNFa ng/L mean (95% CI) | baseline | 1.06 | (0.85-1.27) | 1.17 | (1.02-1.31) | 0.08 |
| | 6-weeks | 1.48 | (1.20-1.77) | 1.31 | (1.16-1.46) | 0.85 |
| IL-1 ng/L mean (95% CI) | baseline | 6.01 | (4.73-7.28) | 5.80 | (5.13-6.46) | 0.75 |
| | 6-weeks | 7.38 | (6.23-8.54) | 6.93 | (6.17-7.70) | 0.80 |
| IL-6 ng/L mean (95% CI) | baseline | 15.35 | (12.80-17.90) | 13.92 | (12.06-15.77) | 0.41 |
| | 6-weeks | 18.71 | (15.38-22.04) | 16.73 | (15.07-18.39) | 0.62 |

TABLE 3
P-value for genetic associations with change in serum cytokine levels uncorrected and after
correction for multiple tests using the false discovery rate (FDR) method. All statistical tests are
corrected for baseline levels, age, BMI and smoking status. P-values under 0.05 are highlighted in
bold typeface.

| | change in TNFα | | change in IL6 | | change in IL1 | |
|---|---|---|---|---|---|---|
| | | FDR | | FDR | | FDR |
| | | corrected p- | | corrected p- | | corrected p- |
| Polymorphism | p-value | value | p-value | value | p-value | value |
| TNF - 308 | 0.86 | | 0.71 | | 0.14 | |
| TNF -238 | 0.001 | 0.016 | 0.59 | | 0.18 | |
| LTA | 0.17 | | 0.52 | | 0.79 | |
| IL10 -1082 | 0.019 | 0.171 | 0.09 | | 0.0005 | 0.016 |
| IL10 -819 | 0.19 | | 0.18 | | 0.0042 | 0.053 |
| IL10 -592 | 0.12 | | 0.21 | | 0.0004 | 0.025 |
| NRAMP vntr | 0.36 | | 0.98 | | 0.67 | |
| IL6 -174 | 0.46 | | 0.15 | | 0.21 | |
| IL6 -634 | 0.08 | | 0.56 | | 0.21 | |
| eNOS 894 | 0.60 | | 0.30 | | 0.43 | |
| IL1A-889 | 0.62 | | 0.29 | | 0.45 | |
| ILB-511 | 0.99 | | 0.21 | | 0.64 | |
| IL4 vntr | 0.08 | | 0.74 | | 0.89 | |
| PPARG 12 | 0.78 | | 0.68 | | 0.56 | |
| MTHFR 677 | 0.98 | | 0.09 | | 0.56 | |
| MTHFR 1298 | 0.69 | | 0.56 | | 0.56 | |
| SOD2 -28 | 0.99 | | 0.042 | 0.331 | 0.33 | |
| GSTM1 Null | 0.42 | | 0.51 | | 0.65 | |
| GSTT1 Null | 0.82 | | 0.21 | | 0.42 | |
| GSTP1 341 | 0.41 | | 0.13 | | 0.19 | |
| GSTP1 313 | 0.51 | | 0.0009 | 0.019 | 0.012 | 0.126 |

What is claimed is:

1. A method for predicting the change in the level of at least one pro-inflammatory cytokine in an individual due to alpha tocopherol supplementation, the method comprising:
analyzing a sample obtained from the human for the presence of one or more genetic variations in at least one gene correlated with a change in serum pro-inflammatory cytokine levels;
detecting the genotype of the at least one gene correlated with a with a change in serum pro-inflammatory cytokine levels; and
predicting an outcome of the change based on the correlation.

2. The method of claim 1, wherein the at least one pro-inflammatory cytokine is selected from the group consisting of TNFα, IL-6, IL-1, and combinations of the foregoing.

3. The method of claim 1, wherein the at least one gene correlated with a change in serum pro-inflammatory cytokine levels is selected from the group consisting of TNF, IL10, SOD2, GSTP1, and combinations of the foregoing.

4. The method of claim 1, wherein the method comprises:
detecting the genotype of the at least one gene selected from the group consisting of:
the TNF genotype at position −308;
the TNF genotype at position −238;
the IL 10 genotype at position −592;
the IL10 genotype at position −1082;
the IL10 genotype at position −819;
the IL10 genotype at position −592;
the IL10 genotype at position −1082;
the SOD2 genotype at position −28;
the GSTP1 genotype at position 313; and
combinations of the foregoing; and
predicting an outcome selected from the group consisting of
an increase in serum levels of TNFα when IL10-1082 SNP has a genotype of AG;
an increase in serum levels of TNFα when IL10-1082 SNP has a genotype of AA;
an increase in serum levels of TNFα when the TNF-238 SNP has a genotype of GG;
a decrease in serum levels of TNFα when the TNF-238 SNP has a genotype of GA;
a decrease in serum levels of IL-6 when the GSTP1 313 SNP has a genotype of GG;
an increase in serum levels of IL-6 when the GSTP1 313 SNP has a genotype of AG;
an increase in serum levels of IL-6 when the GSTP1 313 SNP has a genotype of GG;
an increase in serum levels of IL-6 when the SOD-28 SNP has a genotype of TT;
a decrease in serum levels of IL-1 when the GSTP1 313 SNP has a genotype of GG;
an increase in serum levels of IL-1 when the IL10-819 SNP has a genotype of CC; and
an increase in serum levels of IL-1 when the IL10-1082 SNP has a genotype of GG.

5. The method of claim 1, wherein the level of at least one pro-inflammatory cytokine is a serum level.

6. The method of claim 1, wherein the alpha tocopherol supplementation is about 75 IU/day to about 600 IU/day.

7. The method of claim 1, wherein the alpha tocopherol supplementation is daily for six weeks.

8. The method of claim 1, wherein the gene correlated with a with a change in serum pro-inflammatory cytokine levels is correslated using the false discovery rate (FDR) method.

9. The method of claim 4, wherein said genotpye is determined as part of panel of at least 2 genes that have one or more alleles selected from the group consisting of TNF, IL10, SOD2, GSTP1, and combinations of the foregoing; wherein other genes are selected from methylene-metra-hydro-folate-reductase (MTHFR); methionine synthase reductase (MS-MTRR); methionine synthase (MTR); cystathionine beta synthase (CBS); Manganese superoxide dismutase (MnSOD); superoxide dismutase 3 (SOD3); glutathione S-transferase M1 (GSTM1); glutathione S-transferaseT1 (GSTT1); glutathione S-transferase pi (GSTP1); apolipoprotein C-III (APOC3); apolipoprotein A-V (APOA5); cholesteryl ester transfer protein (CETP); ipoprotein lipase (LPL); endothelial nitric oxide synthase (eNOS); angiotensin converting enzyme gene (ACE); vitamin D receptor (VDR); collagen type 1 alpha 1 (COL1A1); peroxisome proliferator-activated receptor gamma 2 (PPAR-γ2); epoxide hydrolase I (EPHX1); hepatic lipase (LIPC); paraoxonase 1 (PON1); alcohol dehydrogenase IB (ADH1B); alcohol dehydrogenase IC (ADH1C); angiotensinogen (AGT); cytochrome P450 1A1 (CYP1A1); cytochrome P450 1A2*1B (CYP1A2_{—}1B); cytochrome P450 1A2*1E (CYP1A2_{—}1E); and cytochrome P450 1A2*1F (CYP1A2_{—}1F).

10. A method for predicting the response of an individual to alpha tocopherol supplementation, which method comprises:
determining the GSTP1 genotype at position 313; and
predicting a greater response to said alpha tocopherol supplementation when the individual is homozygous at the G allele at position 313 than when the individual has another genotype at position 313.

11. In a method of nutrigenetic screening of an individual, the improvement comprising:
determining whether the individual has a GG genotype at position 313 of the GSTP1 gene; and;
recommending a minimum level of vitamin E intake in the diet to the individual where the GG genotype is present.

12. The method claim 10, wherein the minimum level of vitamin E intake is about 75 IU/day to about 600 IU/day.
